(19) United States
(12) Patent Application Publication
(10) Pub. No.: US 2016/0195506 A1
(43) Pub. Date: Jul. 7, 2016

(54) ESTIMATION OF COLD-FLOW PROPERTIES OF REFINERY PRODUCT BLENDS

(71) Applicant: BHARAT PETROLEUM CORPORATION LTD., Mumbai (IN)

(72) Inventors: Rajeev Kumar, Greater Noida (IN); Chithra Viswanath, Greater Noida, (IN); Sanjay Bhargava, Greater Noida (IN); Ravi Kumar Voolapalli, Greater Noida (IN); Sudha Tyagi, Greater Noida (IN); Shekhar R. Kulkarni, Mumbai (IN); Hari Babu Banoth, Mumbai (IN); Pramod Gulati, Mumbai (IN)

(21) Appl. No.: 14/989,159

(22) Filed: Jan. 6, 2016

(30) Foreign Application Priority Data
Jan. 7, 2015 (IN) 64/MUM/2015

(51) Int. Cl. G01N 33/28

(52) U.S. Cl. CPC G01N 33/2811

(57) ABSTRACT

Method(s) and system(s) for estimation of cold-flow properties of refinery product blends are described. The method may include receiving refinery products to be blended and ratios of the refinery products to be used for producing the refinery product blend, and determining whether the refinery products include at least one heavy product. The cold-flow property of the refinery product blend can be estimated based on a first correlation when the refinery products include the at least one heavy product, and based on a second correlation when the refinery products include no heavy product. The first correlation and the second correlation can differ in at least a sign of a coefficient.

### FIELD OF INVENTION

The present invention relates to a method of estimation of cold-flow properties of refinery product blends, and in particular to estimation of cold-flow properties based on type of refinery products being blended.

### BACKGROUND

Crude oil generally refers to a complex mixture of hydrocarbons, which is obtained from geological formations and from which refined petroleum products can be obtained through fractional distillation. Fractional distillation in a refinery is a multi-step process. Each step in the process yields different refinery products, including distillates and residues, at different boiling ranges. The refinery products are typically blended in various ratios to produce commercial products that meet commercial product specifications.

Further, crude oils vary considerably from each other in yields of the refinery products and in properties of the refinery products obtained. Generally, refineries use a blend of crude oils for meeting the demand and specifications of commercial products, and for cost optimization of refinery operations. The types and amounts of crude oils to be purchased are generally selected based on a prediction of the amount and properties of the refinery products that can be obtained from each of the crude oils and an estimation of the properties of the commercial products that can be obtained on blending those refinery products.

### BRIEF DESCRIPTION OF DRAWINGS

The detailed description is provided with reference to the accompanying figures. In the figures, the left-most digit(s) of a reference number identifies the figure in which the reference number first appears. The same numbers are used throughout the drawings to reference like features and components.

FIG. 1 illustrates a properties estimation system, in accordance with an implementation of the present subject matter.

FIG. 2 illustrates a method for cold-flow properties estimation, in accordance with an implementation of the present subject matter.

FIG. 3A illustrates a graphical representation of validation of the cold-flow properties estimation method for pour point estimation for a refinery product blend having no heavy product, in accordance with an implementation of the present subject matter.

FIG. 3B illustrates a comparative graphical representation for validation of the cold-flow properties estimation method for pour point estimation for a refinery product blend having at least one heavy product, in accordance with an implementation of the present subject matter.

### DETAILED DESCRIPTION

The present subject matter relates to estimation of cold-flow properties of refinery product blends. Refinery products can be obtained from fractional distillation of one or more feed oils, such as a crude oil, crude oil blends, synthetic oils, and hydrocarbon mixtures. While the following description uses crude oil as an example of feed oil, it will be understood that any of the other oils may also be used, as would be evident to a person skilled in the art.

There are various varieties of crude oils that are available in the petroleum market, of which Bombay High crude, Arab Light, and Saharan Blend Crude oil are prominent examples. Every crude oil variety differs from the other in terms of composition and properties. Thus, the amount and quality of refinery products, including distillates and residues, that can be produced from every crude oil also differs. Further, to produce commercial products, the refinery products from different crude oils are generally mixed in different ratios to meet the commercial product specifications and for cost optimization. For example, diesel, kerosene, and naphtha cuts obtained from fractional distillation of one or more feed oils may be mixed in various ratios to produce commercial diesel that meets certain specifications, as applicable in a country in which the commercial diesel is to be marketed, at an acceptable cost.

Generally, to select the starting feed oils, various tests or simulations are carried out based on the properties of the feed oils to predict the refinery products that can be obtained from each of the feed oils. Apart from predicting the refinery products, properties of blends of the predicted refinery products are also estimated to ensure that the demand and specifications of commercial products can be met at an optimum cost. An erroneous estimation of the properties of refinery product blends can lead to sub optimal selection of crude oils and can affect the performance and profitability of refineries.

The properties of the refinery product blends that are generally estimated include cold-flow properties, such as pour point, cloud point, and the like. Pour point of an oil generally refers to the lowest temperature at which the oil becomes semi-solid and loses its flow characteristics, i.e., stops flowing. Cloud point of an oil, on the other hand, is the temperature at which a haze or precipitate appears in the oil. The pour point and cloud point of a refinery product blend determines the usability of the refinery product blend in cold weather conditions. Typically, the commercial products obtained from refinery product blends have to meet cold-flow property specifications, such as pour point and/or cloud point specifications, to be commercially marketable. Hence, selection of feed oils also depends on whether the estimated cold-flow properties of blends of predicted refinery products of the feed oils meets the commercial product specifications.

Conventional techniques for estimating cold-flow properties rely on the use of a pre-programmed mathematical correlation between a cold-flow property, such as pour point, of a blend and the cold-flow property of individual refinery products, such as diesel, kerosene, fuel oil, etc., being blended. The mathematical correlation is generally developed from experimental data. For example, the mathematical correlation for pour point can be developed based on experimental data obtained from multiple refinery product blends and their actual pour points.

Typically, the same mathematical correlation is used irrespective of the type of refinery products being blended since the number of refinery product blends that can be created is large and it may not be feasible to develop and use different correlations for different types of refinery product blends. However, this can result in significant differences between an estimated cold-flow property and the actual cold-flow property depending on the type of refinery products being blended. As a result, the selection of feed oils that is dependent on the estimation of cold-flow properties may become sub-optimal in terms of both cost and usability. Further, the refinery products produced from such feed oils may not be usable as predicted to create the commercial products that meet commercial product specifications. Hence, process changes in refinery operations may be required to be able to use such refinery products to produce the commercial products, which can be costly and can consume a lot of time and resources.

In accordance with the present subject matter, systems and methods for estimating cold-flow properties of refinery product blends are described. The systems and methods are used to estimate the cold-flow properties based on the type of refinery products being blended. In one example, a first correlation may be used to estimate the cold-flow property when at least one heavy refinery product is used in the refinery product blend, and a second correlation is used to estimate the cold-flow property when no heavy product is used in the refinery product blend. A heavy product may refer to a refinery product that is obtained from a blend of residue of a fractional distillation process, which has typically initial boiling point (IBP) greater than 360 deg C., and light distillate materials of a fractional distillation process, which has typically IBP less than 360 deg C. Heavy product may include, for example, fuel oil (FO), low sulphur heavy stock (LSHS), vacuum residue oil (VSO), low sulphur fuel oil (LSFO), long residue (LR), vacuum gas oil (VGO), vacuum residue (VR), and the like. Heavy product may also include heavy products having IBP greater than 360 deg C. and obtained from secondary processing units, such as Fluid Catalytic Cracking Unit (FCCU), Catalytic Cracking Unit (CCU), Hydrocracker, Vis-breaker and Delayed Coker Unit (DCU). It will be understood that all such products are included in the term heavy product as used herein.

The first correlation and the second correlation may differ in at least the sign of a coefficient in the correlations. In one example, the first and the second correlations may differ in both sign and magnitude of the coefficient. This is based on the observation that cold-flow properties are typically a non-linear physico-chemical property and are significantly influenced by molecular interactions. Hence while conventional methods may predict the cold-flow properties for refinery product blends that do not have heavy products, when a heavy product is introduced in the refinery product blend, the conventional methods are unable to account for the effect of the heavy product.

In the present subject matter, by using different signed coefficients, such as positive and negative, in different correlations, the systems and methods are able to take into account the effect of heavy products in the variation of cold-flow properties. The cold-flow property estimate thus obtained is significantly more accurate than the conventional methods and thus leads to better selection of feed oils, better refinery operations, increased production of commercial products, and better overall cost and process optimization. Further, since the systems and methods rely on a small number of different correlations, such as two correlations, they are easy to use and less complicated than having multiple different correlations for the different refinery product blends that can be possibly created.

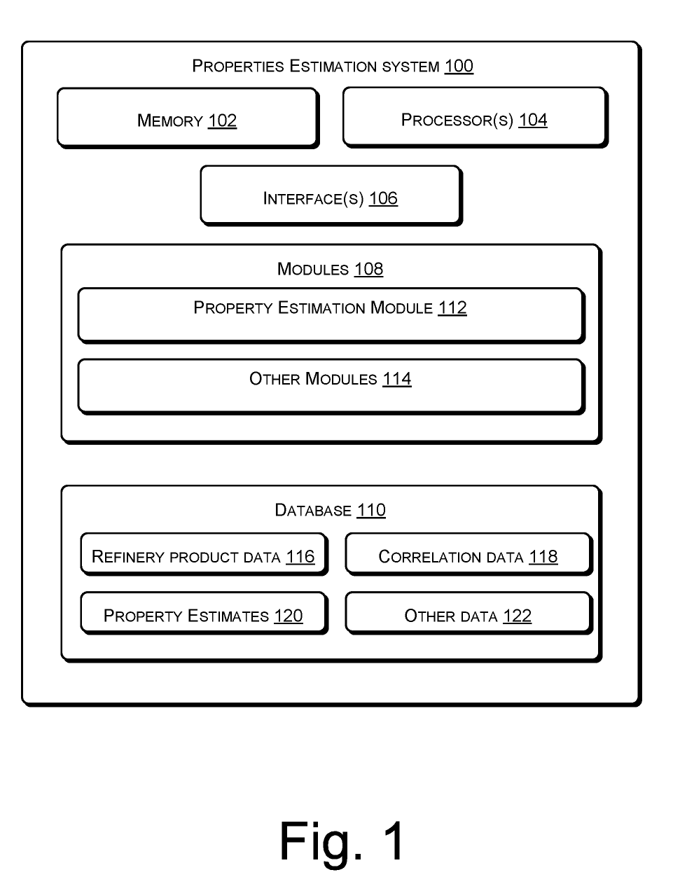

FIG. 1 illustrates various components of a properties estimation system 100, according to an embodiment of the present subject matter. The properties estimation system 100 includes one or more processor(s) 104, one or more interface(s) 106, and a memory, such as a memory 102, coupled to the processor(s) 104. It will be understood that the properties estimation system 100 may be implemented as any suitable computing system known in the art, such as a desktop, a laptop, a server, and the like. The properties estimation system 100 may be interchangeably referred to as system 100 hereinafter.

The memory 102 may include any computer-readable medium known in the art including, for example, volatile memory, such as static random access memory (SRAM) and dynamic random access memory (DRAM), and/or non-volatile memory, such as read only memory (ROM), erasable programmable ROM, flash memories, hard disks, optical disks, and magnetic tapes.

The processor(s) 104 can be a single processing unit or a number of units, all of which could include multiple computing units. The processor(s) 104 may be implemented as one or more microprocessors, microcomputers, microcontrollers, digital signal processors, central processing units, state machines, logic circuitries, and/or any devices that manipulate signals based on operational instructions. Among other capabilities, the processor(s) 104 is configured to fetch and execute computer-readable instructions and data stored in the memory 102.

The functions of the various elements shown in the figures, including any functional blocks labeled as “processor(s)”, may be provided through the use of dedicated hardware as well as hardware capable of executing software in association with appropriate software. When provided by a processor, the functions may be provided by a single dedicated processor, by a single shared processor, or by a plurality of individual processors, some of which may be shared. Moreover, explicit use of the term “processor” should not be construed to refer exclusively to hardware capable of executing software, and may implicitly include, without limitation, digital signal processor (DSP) hardware, network processor, application specific integrated circuit (ASIC), field programmable gate array (FPGA), read only memory (ROM) for storing software, random access memory (RAM), and non volatile storage.

The interface(s) 106 may include a variety of software and hardware interfaces, for example, interfaces for peripheral device(s), such as a keyboard, a mouse, an external memory, and a printer. The interface(s) 106 can facilitate multiple communications within a wide variety of networks and protocol types, including wired networks, for example, local area network (LAN), cable, etc., and wireless networks, such as Wireless LAN (WLAN), cellular, or satellite. For the purpose, the interface(s) 106 may include one or more ports for connecting a number of devices to each other or to another computing system.

In one implementation, processor(s) 104 is coupled to module(s) 108 and database 110. In another implementation, the module(s) 108 and database 110 may reside in the memory 102 and the memory 102 may be coupled to the processor(s) 104. The modules 108, amongst other things, include routines, programs, objects, components, data structures, etc., which perform particular tasks or implement particular abstract data types. The database 110 serves, amongst other things, as a repository for storing data processed, received, and generated by the modules 108, where the data may be fetched by the processor(s) 104.

The modules 108 include a blend property estimation module 112 that, amongst other functions, can estimate the cold-flow properties of refinery product blends, and other modules 114, such as operating system, that supplement the operation of the system 100. The data in the database 110 may include refinery product data 116, correlation data 118, property estimates 120, and other data 122. The refinery product data 116 can include known properties of refinery products. The correlation data 118 can include coefficients and correlations between properties of refinery product blends and refinery products. The property estimates 120 can include estimated properties, including estimated cold-flow properties determined for one or more refinery product blends. The other data 122 includes data generated as a result of the execution of one or more modules. In one example, though not shown herein, some or all of the data in the database 110, such as the refinery product data 116, can be stored in a separate database that can be accessed by the properties estimation system 100.

In one example, the modules 108 may further include, for example, a correlation determination module (not shown in figure) and a refinery product prediction module (not shown in figure). In another example, the correlation determination module and the refinery product prediction module may be hosted on one or more different computing systems, and the results of operations of the correlation determination module and the refinery product prediction module may be used by the blend property estimation module 112 for estimating cold-flow properties of refinery product blends in accordance with various examples of the present subject matter.

In operation, the property estimation module 112 may receive inputs related to refinery products that may be produced on refining of one or more feed oils based on, for example, a refinery product prediction, actual refinery products produced, user inputs, or a database. The property estimation module 112 may further receive inputs identifying the refinery products to be blended and the ratio in which the refinery products are to be blended. This may be an iterative process. For example, the property estimation module 112 may first receive a default product blend ratio related to the suitability to meet product specification and cost economics as input, and may estimate properties of the blended product. In case the product blend does not meet the desired product specification or costs then a new product blend ratio may be received as input and so on till a suitable product blend ratio is identified.

To estimate the properties of the blended product, on receiving the inputs, the property estimation module 112 may determine whether the identified refinery products to be blended include at least one heavy oil or heavy product. A heavy product can be understood as a refinery product obtained from a residue of a fractional distillation process and can include, for example, fuel oil (FO), low sulphur heavy stock (LSHS) oil, low sulphur fuel oil (LSFO), vacuum gas oil (VGO), long residue (LR), vacuum residue (VR), etc. For this, the property estimation module 112 may fetch refinery product data 116 from the database 110 and compare the properties of the products received as input with the properties stored in the refinery product data 116 to determine if at least one heavy product is present in the received input.

In case the identified refinery products include at least one heavy product, the property estimation module 112 may estimate the cold-flow property of the refinery product blend based on a first correlation. On the other hand, when the refinery products include no heavy product, the property estimation module 112 may estimate the cold-flow property of the refinery product blend based on a second correlation. Further, the first correlation and the second correlation differ in at least a sign of a coefficient. In one example, the first correlation and the second correlation differ in both sign and magnitude of a coefficient. The first and second correlation and respective coefficients may be fetched from the correlation data 118 for estimating the cold-flow property. In one example, the first and the second correlation and respective coefficients may be predetermined and stored in the correlation data 118, for example, by any of the other modules 114 or using a different computing system. An example method of determination of the first and the second correlation and respective coefficients is discussed below and example correlation equations are provided below.

In one implementation, to determine the first and second correlations, pour point of sample refinery product blends having different ratios of heavy product, from 0% to 100%, can be measured using standard pour point measurement techniques (ASTM D97 and D5949) and the correlations can be determined based on regression analysis of the measured pour points and calculated pour point indices of the sample blends. Accordingly, two correlations can be determined, one for estimating the pour point when there is no heavy product in the blend and one for estimating the pour point when there is at least one heavy product in the blend. As is known the pour point index (PPI) of a blend can be calculated based on the pour point indices of the constituent refinery products and the weight fraction of each of the constituent products. The determination of the PPI can be thus independent of the type of the refinery product being blended, i.e., irrespective of whether the blend includes a heavy product. The PPI thus calculated can be used in the first and second correlations for determining the pour point of the blend.

For example, for estimating the pour point of a refinery product blend including a heavy product, the following correlation as given in equation 1 may be used as the first correlation:

PPI_{B,H}=EXP(A₁+A₂*(32+1.8*PP_{B,H})) (Eq. 1)

where PPI_{B,H} is the Pour Point Index (PPI) of the Blend having a heavy product;

PP_{B,H} is the Pour Point (PP) of the Blend having a heavy product, in deg Celsius;

A₁ is a constant determined from regression. For example, A₁ can be about 2.19; and

A₂ is the first coefficient determined from regression. For example, A₂ can be about −0.010. Here, the PPI_{B,H} can be determined from the pour point PPIᵢ of the individual refinery products being blended, based on equation 2 given below:

PPI_{B,H}=Σ(PPIᵢ*Xᵢ) (Eq. 2)

where, Xi=weight fraction of component i in the blend

Further, for estimating the pour point of a refinery product blend not including any heavy product, the following correlation as given in equation 3 may be used as the second correlation:

PPI_{B,L}=EXP(A₁+A₃*(32+1.8*PP_{B,L})) (Eq. 3)

where PPI_{B,L} is the Pour Point Index (PPI) of the Blend having no heavy product;

PP_{B,L} is the Pour Point of the Blend having no heavy product, in deg Celsius;

A₁ is a constant determined from regression. For example, A₁ is about 2.19; and

A₃ is the second coefficient determined from regression. For example, A₃ is about 0.035. Here, the PPI_{B,L} can also be determined from the PPIᵢ of the individual refinery products being blended, based on equation 2 given above.

It can be observed that the first and second correlations differ at least in the sign of the coefficient of PP_{B}. Further, it can be observed that the correlations are of the following form:

PPI_{B}=EXP(C+Kᵢ*(32+1.8*PP_{B})) (Eq. 4)

(or) PP_{B}=((LN(PPI_{B})−C)/Kᵢ)−32)/1.8 (Eq. 5)

where, C is a constant, and can lie in the range 2-3, for example, C=A₁=about 2.19 as above.

Kᵢ is a coefficient of varying sign.

In the above example, K₁ lies in the range −0.01 to −0.10 is the first coefficient when the blend includes a heavy product. For example, K₁=A₂=about −0.010 in Eq. 1. K₂ lies in the range 0.01 to 0.10 is the second coefficient when the blend includes no heavy product. For example, K₂=A₃=about 0.035 in Eq. 3.

Accordingly, in one implementation, the property estimation module 112 may use the correlation as per equation 5 to estimate the pour point of the blend, based on the PPI of the blend determined as per equation 2 given above, using first and second coefficients. In another implementation, the property estimation module 112 may use equation 1 as the first correlation having a negative coefficient and equation 3 as the second correlation having a positive coefficient, to determine the pour point of the blend depending on whether at least one heavy product is present in the blend or not. Further, before determining the pour point of the blend, the property estimation module 112 may first determine the pour point index of the blend using equation 2, as mentioned above.

While the description of the present subject matter has been provided with reference to estimation of pour point, it can also be used for estimation of other cold-flow properties, albeit with a few variations, as would be understood by a person skilled in the art. For example, to determine the cloud point of the blend, equations similar to equations 1-5 can be determined based on empirical studies. In one example, the correlations can be determined by measuring the cloud point of the blend using different ratios of heavy product in the blend, starting from 0% to 100%, and using regression analysis to determine the equations for estimating the cloud point with no heavy product in the blend and with at least one heavy product in the blend.

The cold-flow properties thus estimated, using the first and second correlations or coefficients of varying sign, are more accurate than conventionally determined cold-flow properties. The estimated cold-flow property of the refinery product blend can be then provided to a user or to another computing system for feed oil selection and refinery process optimization As a result, the selection of crude oils and processing parameters, and operations of the refinery can be better controlled.

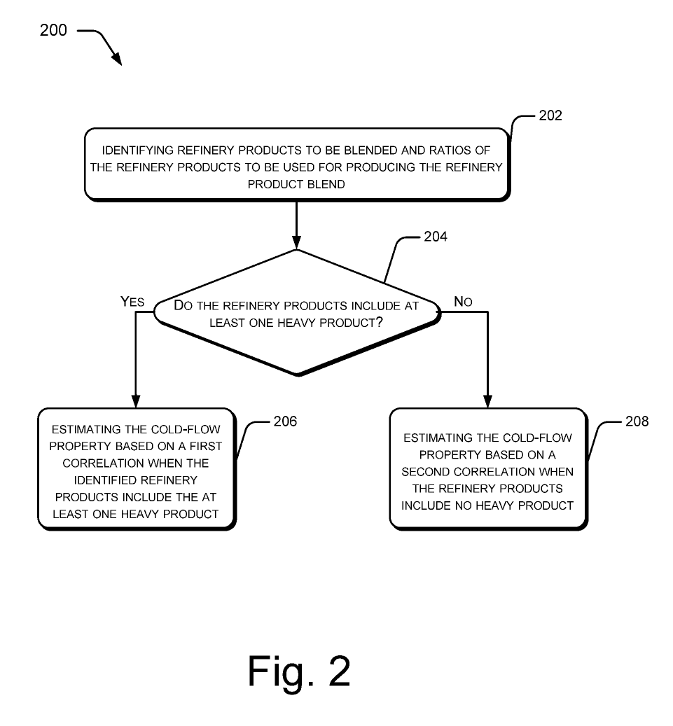

FIG. 2 illustrates an example method 200 for cold-flow properties estimation, in accordance with an implementation of the present subject matter. The method 200 may be described in the general context of computer executable instructions. Generally, computer executable instructions can include routines, programs, objects, components, data structures, procedures, modules, functions, etc., that perform particular functions or implement particular abstract data types. The method may also be practiced in a distributed computing environment where functions are performed by remote processing devices that are linked through a communications network.

The order in which the method blocks are described is not intended to be construed as a limitation, and any number of the described method blocks can be combined in any order to implement the method 200, or an alternative method. Additionally, individual blocks may be deleted from the method 200 without departing from the scope of the subject matter described herein. Furthermore, the method 200 can be implemented in any suitable hardware, software, firmware, or combination thereof. For example, the method 200 may be implemented based on computer-readable instructions stored in a non-transitory computer readable medium, such as the memory 102. The method 200 is explained with reference to the properties estimation system 100 as an example and without limitation.

At block 202, refinery products to be blended and ratios of the refinery products to be used for producing the refinery product blend are received as input.

At block 204, it is determined whether the identified refinery products include at least one heavy product, based on refinery product data 116. If the refinery products include at least one heavy product, the method 200 proceeds to block 206, else, the method 200 proceeds to block 208.

At block 206, the cold-flow property of the refinery product blend is estimated based on a first correlation when the identified refinery products include the at least one heavy product. Whereas, at block 208, the cold-flow property of the refinery product blend is estimated based on a second correlation when the refinery products include no heavy product. The first correlation and the second correlation differ in at least a sign of a coefficient. The estimation of cold-flow property at block 206 and at block 208 is performed using correlation data 118. The estimated cold-flow property of the refinery product blend can be then provided to a user or to another computing system for feed oil selection and refinery process optimization.

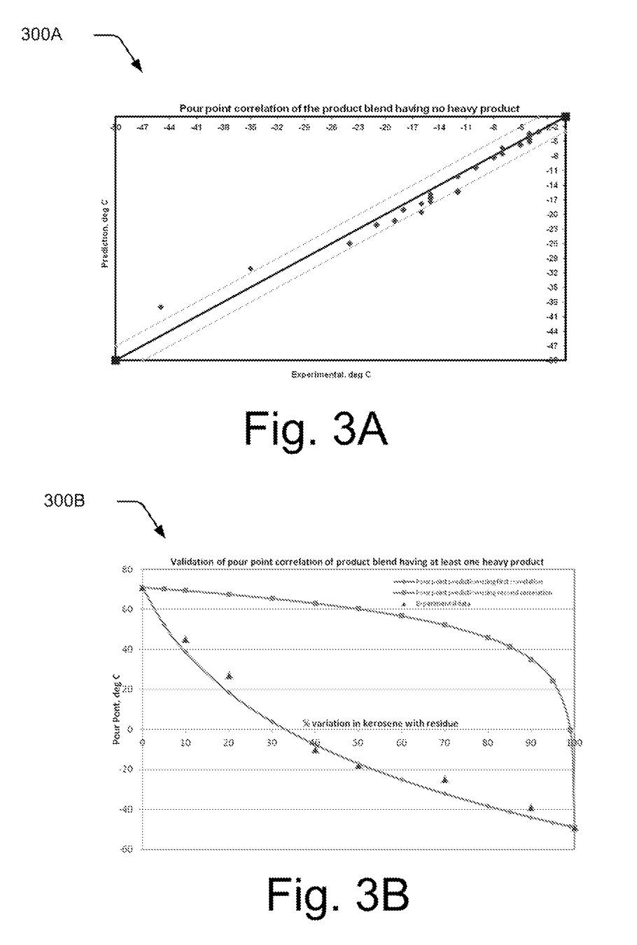

FIG. 3A illustrates a graphical representation of validation of the cold-flow properties estimation method for pour point estimation for a refinery product blend having no heavy product, in accordance with an implementation of the present subject matter. Graph 300A shows the relationship between predicted and experimentally measured pour point temperatures for a refinery product blend having no heavy product using the second correlation, in particular, equation 3 discussed above. As can be seen, the predicted pour point temperature is in a range of ±3° C. with respect to the measured pour point temperature.

FIG. 3B illustrates a comparative graphical representation for validation of the cold-flow properties estimation method for pour point estimation for a refinery product blend having at least one heavy product, in accordance with an implementation of the present subject matter. Graph 300B shows the relationship between predicted and experimentally measured pour point temperatures for a refinery product blend having at least one heavy product using the first correlation, in particular, equation 1 discussed above. In this example, the refinery product blend included fuel oil (FO) and/or LSHS/LSFO in different ratios with kerosene. As can be seen from 300B, the predicted pour point temperature is in a range of ±6° C. with respect to the measured pour point temperature. This is in contrast to conventional techniques of using a single pour point correlation, i.e., the second correlation alone, irrespective of the presence of a heavy product, where the predicted temperature could vary up to ±30° C. As is seen in 300B, the use of the first correlation for predicting pour point when at least one heavy product is present is much more accurate and the result is closer to the experimentally measured pour point than when the second correlation is used.

Although implementations for estimation of cold-flow properties of refinery product blends have been described in language specific to structural features and/or methods, it is to be understood that the appended claims are not necessarily limited to the specific features or methods described. Rather, the specific features and methods are disclosed as example implementations for estimation of cold-flow properties of refinery product blends.

I/We claim:

1. A method for estimating a cold-flow property of a refinery product blend for feed oil selection and refinery process optimization, the method comprising:
receiving, by a processor, inputs including properties of refinery products to be blended and ratios of the refinery products to be used for producing the refinery product blend;
determining, by the processor, whether the refinery products include at least one heavy product based on the received properties of refinery products and refinery product data fetched from a database;
estimating, by the processor, the cold-flow property of the refinery product blend based on a first correlation when the refinery products include the at least one heavy product, wherein the first correlation is fetched from correlation data in the database;
estimating, by the processor, the cold-flow property of the refinery product blend based on a second correlation when the refinery products include no heavy product, wherein the second correlation is fetched from the correlation data in the database, wherein the first correlation and the second correlation differ in at least a sign of a coefficient; and
providing, by the processor, the estimated cold-flow property of the refinery product blend for feed oil selection and refinery process optimization.

2. The method as claimed in claim 1, the cold-flow property is one of pour point and cloud point.

3. The method as claimed in claim 1, wherein, when the cold-flow property is pour point, the first and second correlation provide correlations between a pour point index of the refinery product blend and the pour point of the refinery product blend.

4. The method as claimed in claim 3, comprising determining, by the processor, the pour point index of the refinery product blend based on pour point indices of the refinery products and weight fractions of the refinery products in the refinery product blend.

5. The method as claimed in claim 1, wherein when the cold-flow property is pour point, the first correlation has a negative coefficient and the second correlation has a positive coefficient.

6. A system for estimating a cold-flow property of a refinery product blend for feed oil selection and refinery process optimization, the system comprising:
a processor;
a database comprising refinery product data and correlation data; and
a properties estimation module coupled to the processor and the database to:
receive, as input, properties of refinery products to be blended and ratios of the refinery products to be used for producing the refinery product blend;
determine whether the refinery products include at least one heavy product based on the input and the refinery product data;
retrieve a first coefficient from the correlation data when the identified refinery products include the at least one heavy product and retrieve a second coefficient from the correlation data when the identified refinery products include no heavy product; and
estimate the cold-flow property of the refinery product blend based on the retrieved coefficient, a correlation between the cold-flow property and a cold-flow property index that includes the coefficient, and the refinery product data.

7. The system as claimed in claim 6, wherein the cold-flow property is one of pour point and cloud point.

8. The system as claimed in claim 6, wherein, when the cold-flow property is pour point, the cold-flow property index is a pour point index of the refinery product blend.

9. The system as claimed in claim 8, comprising determining the pour point index of the refinery product blend based on pour point indices of the refinery products available in the refinery product data and weight fractions of the refinery products in the refinery product blend.

10. The system as claimed in claim 6, wherein when the cold-flow property is pour point, the first coefficient is negative and the second coefficient is positive.
